(19) United States
(12) Patent Application Publication
(10) Pub. No.: US 2010/0136647 A1
(43) Pub. Date: Jun. 3, 2010

(54) METHOD FOR PRODUCTION OF CELL ATTACHMENT AND CULTURE SURFACES

(75) Inventors: Mattias Algotsson, Uppsala (SE); Hans Berg, Uppsala (SE); Asa Bjurling, Uppsala (SE); Christian Kaisermayer, Vienna (AT); Bjorn Noren, Uppsala (SE); Nicholas Thevenin, Saint Cyr (FR); James Van Alstine, Uppsala (SE)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCES AB, Uppsala (SE)

(21) Appl. No.: 12/623,528

(22) Filed: Nov. 23, 2009

(30) Foreign Application Priority Data
Nov. 25, 2008 (SE) 0802474-7

(51) Int. Cl. C12N 11/12; C12N 11/00; C12N 11/10; C12N 11/06

(52) U.S. Cl. 435/179; 435/174; 435/178; 435/181

(57) ABSTRACT

The present invention relates to the field of adherent cell culture. More closely, the invention relates to a method for production of a cell attachment and culture surface, such as a microcarrier, comprising a guanidino-containing ligand, wherein the ligand is coupled via reaction involving a primary amine to the surface which is activated by activation groups such that the final molar ratio of grafted ligand and ungrafted activation groups is above 1.5. Preferably, the ligand density is above 0.5 mmol/g cell culture surface and the remaining activation groups after coupling is less than 0.6 mmol/g cell culture surface. The cell culture surface may be used for various purposes, primarily cell cultivation and virus production.

### CROSS-REFERENCE TO RELATED APPLICATIONS

This application claims priority to Swedish patent application number 0802474-7 filed Nov. 25, 2008; the entire disclosure of which is incorporated herein by reference.

### FIELD OF THE INVENTION

The present invention relates to the field of adherent cell culture. More closely, the invention relates to a method for production of a cell attachment and culture surface, such as a microcarrier, comprising a guanidino-containing ligand, wherein the ligand is coupled via a primary amine to an activated microcarrier. The microcarrier may be used in, for example, cell cultivation and virus production.

### BACKGROUND OF THE INVENTION

Cell culture techniques are vital to the study of animal cell structure, function and differentiation and for the production of important biological materials, such as virus vaccines, enzymes, hormones, antibodies, interferons, nucleic acids and virus vectors for gene therapy. Another important area for cell culture and therapy is cell expansion from a small to a large cell population.

Most mammalian cells and many other cells are anchorage-dependent and need suitable surfaces on which to grow. Culture of adherent cells on the surfaces of bottles, flasks or other containers produces yields limited by available surface area.

Microcarrier culture helps to make it possible to achieve a high yield culture of anchorage-dependent cells. In microcarrier culture cells typically grow as monolayers on the surface of small spheres, which are usually suspended in culture medium by gentle stirring. Use of microcarriers in simple suspension culture systems makes it possible to achieve yields of several million cells per millilitre. In addition such systems are easily scalable. Cells can be grown in large bioreactors or smaller bottles or flasks or even on carrier beads in microtitre plates or in columns (perfusion culture). The microcarriers can be made of various biocompatible materials such as agarose, dextran, cellulose or polyethylene polymers.

In order to more closely mimic in vivo conditions, and therefore cell attachment and growth, microcarriers are often provided with an animal protein-derived coating, such as a coating of collagen in the form of porcine or bovine gelatin. Leakage of animal protein from conventional microcarrier media may be a problem, especially in the production of cells and vaccines for therapy. It is thus desirable to have an animal protein free microcarrier product replacing animal protein containing products, such as porcine collagen-coated microcarriers.

Cells are cultured on a wide variety surfaces for a large number of reasons including biocatalysis using cell enzymes, bioproduction of cells or cell components or cell products, therapy related culture of cells or cell products, cell based sensing and high throughput screening. All such applications require cell culture surfaces which promote target cell attachment and culture and, in some cases, also allow for effective cell removal by enzymatic or other approaches. Many of these applications require surface attached ligands (or other surface treatments) to improve surfaces for cell interactions. Some benefit from the ability to pattern or otherwise control the topographical presentation of ligands and related attachment of cells. Such ligands must be simple, inexpensive, biocompatible, and readily attached to a variety of surfaces by simple chemical and production methods. The cell culture surfaces should not be of animal origin and should function with variety of target cells (e.g. Vero and other cells used in bioproduction, stem cells for cell therapy and drug screening, etc.)

US 2006-0252152 A1 describes a microcarrier onto the surface of which a cationic compound has been immobilised via a guanidine group. The microcarrier is capable of cell attachment, e.g. via charge-based interaction, and is used as a support in the culture of cells. Said compound may comprise one or two amino acids, such as L-arginine (Arg) or a dipeptide. The invention also relates to a method of preparing a polycationic microcarrier, which method comprises to immobilise a compound that comprises at least one guanidine group to an epoxide-activated substrate. Not all guanidine containing groups are biocompatible; some have well known bacteriostatic or cell toxic properties. (e.g. Anticancer Drugs vol. 15, pp. 45-54, 2004. Development and characterization of two human tumor sublines expressing high-grade resistance to the cyanoguanidine CHS 828. Joachim Gullbo, Henrik Lövborg, Sumeer Dhar, Agneta Lukinius, Fredrik Oberg, Kenneth Nilsson, Fredrik Björkling, Lise Binderup, Peter Nygren, Rolf Larsson). Even some amino acid analogues can be cytotoxic. The L-arginine analogue L-canavanine induces apoptotic cell death in some cells (e.g. Biochemical and Biophysical Research Communications, Vol. 295, pp. 283-288, 2002. Arginine antimetabolite L-canavanine induces apoptotic cell death in human Jurkat T cells via caspase-3 activation regulated by Bcl-2 or Bcl-xL. Myung Ho Jang, Do Youn Jun, Seok Woo Rue, Kyu Hyun Han, Wan Park, Young Ho Kim).

The above examples refer to chemicals in solution; when attached or otherwise associated with a surface such chemicals may or may not exhibit cytotoxic or other properties that inhibit cell culture. That depends on many factors including the method and path of surface attachment. In some cases surface associated guanidine containing substances may be cytotoxic. Thus U.S. Pat. No. 6,929,818 (Methods and clinical devices for the inhibition or prevention of mammalian cell growth) describes inhibition of mammalian cell growth at biomedical surfaces associated with at least one biguanide group.

The ability of surface immobilization to alter the cytocompatibility of ligands can be further illustrated by hydroxyl group containing substances. In general hydroxyl containing substances are nonreactive and quite benign. However a large body of experimental data suggests that when various surfaces are coated with hydroxyl containing substances they do not support significant protein adsorption or cell attachment and subsequent cell growth (e.g. Langmuir, Vol. 13, pp. 3404-3413, 1997. Endothelial cell growth and protein adsorption on terminally functionalized, self-assembled monolayers of alkanethiolates on gold. Caren D. Tidwell, Sylvie I. Ertel, and Buddy D. Ratner, Barbara J. Tarasevich, Sundar Atre, and David L. Allara).

Given the above it would be desirable if a relatively simple, and robust chemical synthetic path for generation of cell culture surfaces could be identified.

### SUMMARY OF THE INVENTION

The present invention provides a method for production of cell attachment and culture surfaces enabling controlled cell growth and high yield of cell culture. The method provides for covalent coupling of guanidine containing ligands, such as arginine and chemically related substances such as diarginines and other dipeptides, in a manner that allow for generation of cell culture surfaces. The cell cultivation surfaces produced by the method of the invention are shown to be suitable for a wide variety of ligand and cell types. The present inventors have identified how surface activation, further modification and ligand density affect the performance of such cell culture surfaces. In doing so they have potentially identified routes to generation of confluent as well as patterned culture surfaces.

Examples of cell culture surfaces include cell carrier beads such as CYTODEX™ beads, or the inside surfaces of rectangular (cuboidal) or round plastic or glass flasks, or plastic or glass microscope slides or well slides, microtitre plates, as well as the surfaces of chips or sensors which monitor cellular responses. They can also include various prosthetic or other biomaterials related structures (e.g. Biomaterials, Vol. 29, pp. 2802-2812, 2008. Three-dimensional polymer scaffolds for high throughput cell-based assay systems, Ke Cheng, Yinzhi Lai, William S. Kisaalita*).

Cell culture microcarriers are preferred in cases where total cell production per liter of culture fluid is a concern. They may also be preferred in some cases where their materials and surface features more closely mimic natural biological surfaces (for a discussion see above ref in Biomaterials Vol. 29). In many cases the materials are modified, to enhance cell binding and growth, with various surface treatments including cell binding ligands or proteins, e.g. with gelatin protein in the case of CYTODEX™ 3 beads. It should be noted that cell binding is only the first phenomena involved in cell growth. Other phenomena including cell spreading, cell mitosis etc. However many applications, especially analytical or high throughput screening applications, only require that cells bind to surfaces (i.e. do not need to grow) and that cells are not significantly affected by the localisation.

Other advantages of the invention are that the cell culture surfaces can be produced as animal origin free (AOF) and give a high virus productivity.

In a first aspect the invention relates to a method for production of a cell attachment and culture surface comprising a biocompatible guanidine-containing ligand, wherein the ligand is coupled via reaction involving a primary amine to the surface which is activated by activation groups such that the final molar ratio of grafted ligand and ungrafted activation groups is above 1.5.

Preferably, and still keeping the above mentioned ratio of 1.5, the ligand density in itself should also be above 0.5 mmol/g cell culture and the density of activation groups remaining after coupling is less than 0.6 mmol/g cell culture surface. The cell culture surface is preferably a microcarrier based on a natural polymer, such as dextran, starch, cellulose. It is to be understood that these mmol/g concentrations relate to surface concentrations calculated based on reactive surface area of dextran-based microcarriers, such as SEPHADEX™ G50 (GE Healthcare Bio-Sciences AB, Uppsala, Sweden) and may have to be adjusted for carriers with other surface areas.

The ligand may be Arg, agmatine, guanosine, guanidine, adenosine or an analogous substance, or derivatives thereof, or combinations thereof. Also, the ligand may comprise a dipeptide including at least one Arg.

The activation groups are preferably selected from allyl, epoxide or glycidoxyl groups.

In some cases cells may not colonize the entire volume of the carrier and thus the microcarrier may be readily provided with other properties such as magnetic properties to facilitate handling of the microcarriers and/or to control localization, or reporter properties based on imaging, fluorescent, radioactive or other groups.

Preferably, the surface or microcarrier is coated with an animal protein-free coating.

In a further embodiment, the microcarrier may be made of biodegradable material.

In a second aspect, the invention relates to microcarriers produced according to the above methods.

In a third aspect, the invention relates to use of the microcarriers for cell attachment including cultivation.

A further use of the microcarriers is for virus/vaccine production.

There are several other potential uses of surfaces which present arginine or similar ligands in a manner which binds cells, and judging from the ability of such bound cells to be cultured, in a confluent or patterned surface distribution, in a manner that does not significantly alter native cell function. Such uses may include slide, sensor or other flat surfaces used to bind cells for analytical applications such as high throughput screening.

The microcarriers may also be used for diagnostic purposes, such as culture and testing of pathogenic cells for drug sensitivity.

A further use is to promote biocompatible surfaces for implant, prosthetic, drug delivery, or other in vivo medical applications.

Another use is to construct a biosensor or biochip dependent on cell attachment in a manner allowing for viable cells. The cells may be eukaryotic or prokaryotic. Alternatively, the biosensor is used for virus or other bioparticles.

The ligand is coupled to an activated surface via a primary amine which provided suitable culture surfaces (Table 1). Preferably the ligand is coupled to an glycidoxyl group activated surface such that ligand density supports significant cell attachment and growth, which are not otherwise inhibited by the presence of unreacted glycidoxyl groups or hydroxyl groups (arising from the natural hydrolysis of such glycidoxyl groups).

### BRIEF DESCRIPTION OF THE DRAWINGS

FIG. 1 shows growth curves for Vero cells grown in spinner flasks on microcarriers produced according to the invention.

FIG. 2 shows growth curves for MDCK cells grown in spinner flasks on microcarriers produced according to the invention.

FIG. 3 shows growth curves for hMSC cells grown on microcarriers produced according to the invention.

FIG. 4 shows cell morphology of Vero cells grown on microcarriers produced according to the invention.

FIG. 5A-5C shows the effect of Ligand density on an allylated gel with 112 umol allyl/ml gel before ligand coupling (FIG. 5A), Uncoupled allyl groups (FIG. 5B) and the Ratio of covalently coupled arginine to uncoupled allyl groups (FIG. 5C) the latter which are expected to then be hydrolysed to two hydroxyl groups, on the cell growth of Vero cells.

FIG. 6A-6B shows the total and specific virus productivity of Vero (FIG. 6A) and MDCK cells (FIG. 6B) grown on the microcarriers of the invention, compared to commercial CYTODEX™ 1 and CYTODEX™ 3, when infected with influenza virus Productivity is measured in terms of assay units of hemagglutinin (HA) and HA units per cell.

### DETAILED DESCRIPTION OF THE INVENTION

The invention will now be described more closely in association with the drawings and some non-limiting examples.

The present inventors realized the importance of two points in regard to development of cell carrier ligands. Firstly that ligands based on naturally occurring chemical structures (e.g. guanidines) or biochemical substances (e.g. arginine amino acid or arginine containing peptides) may not necessarily be effective promoters of cell culture. Secondly that chemical structure modifications related to covalently linking such structures or biosubstances to surfaces may render them ineffective. One reason for the latter that cell culture on carriers is a complicated matter involving several distinct and complex cell physiology related stages including cell adsorption followed by cell attachment then spreading, prior to growth and division. Cell spreading appears medicated in part by proteins excreted by cells to create an extracellular matrix which conditions the surfaces they are attached to.

The ligands listed in Table 1 were used in the methods of the invention for production of microcarriers which were capable of supporting cell attachment and growth. They included arginine derivatives, di-arginines, hydroxyl and ester group modified arginine analogues, and other related substances. They also included mixtures of such ligands.

TABLE 1

| Ligand | Structure |
|---|---|
| Agmatine | 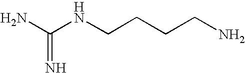 |
| Arg + Lys | Mixture of two ligands 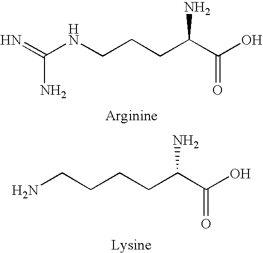 |
| Arginine | 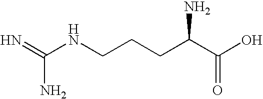 |
| H-Arg-Lys-OH | 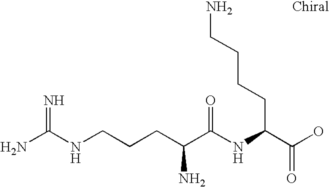 |
| H-Arg-NH2 | 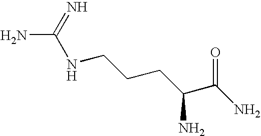 |
| H-Arg-Oet | 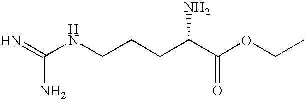 |
| H-Arg-Arg-OH | 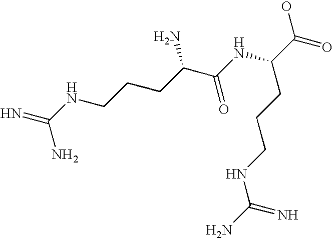 |

As appears from the Table 1, all selected ligands contain at least one primary amino group and a guanidine group. Using different activated gels and different reaction conditions the ligand density of each ligand can be adjusted.

### EXAMPLES

The present invention will be described in more detail by way of examples, which however are in no way intended to limit the scope of the present invention as defined by the appended claims. All references given below or elsewhere in the present specification are hereby included herein by reference.

### Activation of Microcarriers

Activation of microcarriers (here exemplified with SEPHADEX™ beads) by allylation:

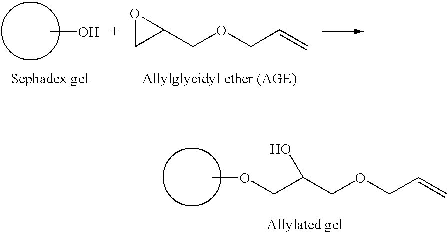

### Allylation Reaction:

SEPHADEX™ G-50 60-87 um was mixed with water in a three-necked flask with stirrer. Na₂SO₄ was added to the flask and was dissolved for 1.5 h at 30° C.
NaOH 50%, NaBH₄ and allyl glycidyl ether (AGE) was added. The slurry was heated to 50° C. and the reaction was continued over night. The reaction was stopped by neutralizing with acetic acid 60%. The gel bead particle was washed with water, ethanol and finally with water.

### Coupling of Ligands to Activated Microcarriers

The different ligands (here exemplified with arginine) can then be coupled to the allylated gel:

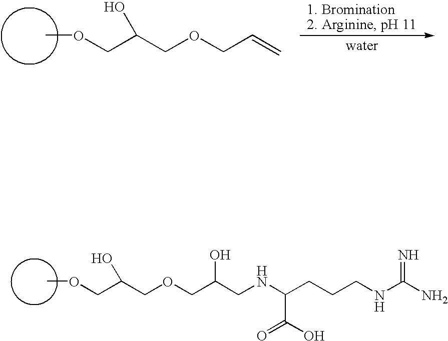

The coupling is done via the primary amine on the C2-carbon of the amino acid Arginine. All ligands used in the invention contain a guanidino group intended for cell attachment and a primary amine intended for coupling to the activated microcarrier.

### Coupling Reaction:

Drained allylated gel was transferred to a beaker and water (approximately the same amount water as the transferred drained gel volume) was added to the gel. During vigorous overhead stirring bromine (pure bromine or bromine water) was added to a consistent yellow colour. After about 5 minutes of stirring sodium formate was added until the gel slurry was completely discoloured and then left stirring for about 15 minutes. The gel was then transferred to a glass filter and vacuum applied until the gel (bead particle) was dry.

The gel was then transferred to a flask and the slurry concentration was adjusted by adding water. Overhead stirring was begun and L-arginine was added to the gel slurry. After stirring for approximately 30 minutes at 55° C. the pH was adjusted with NaOH (50% solution) to around 10. The slurry was then left stirring at 55° C. over night. The reaction was stopped after about 18 hours and the gel washed with 0.9% NaCl, 0.1M NaOAc and finally with 0.9% NaCl.

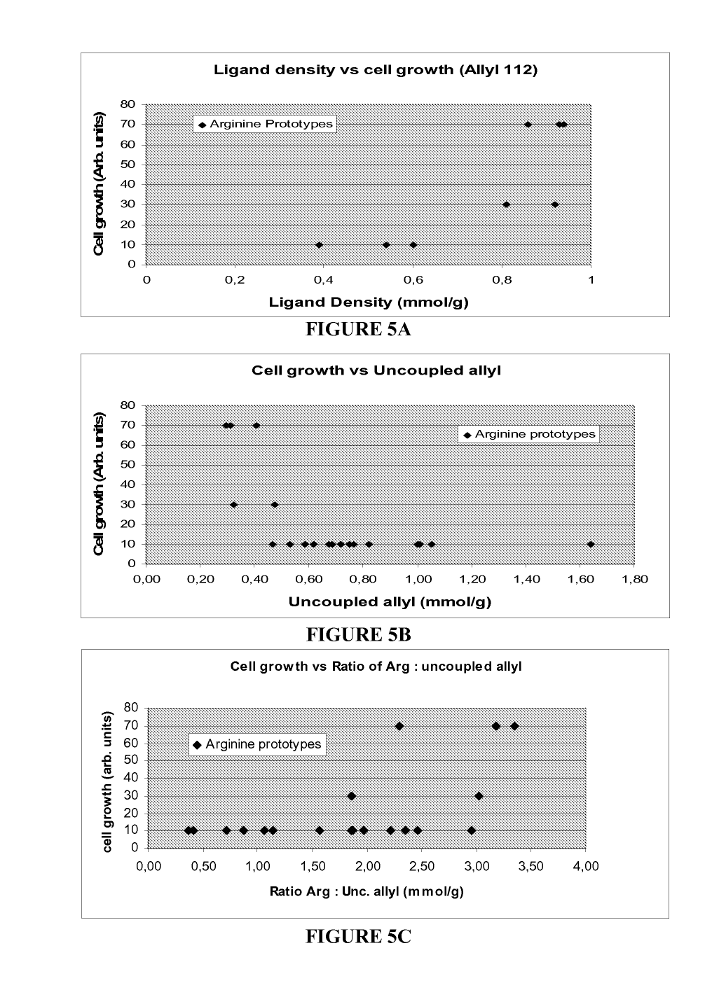

The gel was transferred to a beaker and allowed to sediment for at least 30 minutes. The supernatant was then removed and acetone (approximately 1 gel volume) was added. The slurry was then thoroughly mixed and left for at least 1 h. This procedure was then repeated with a new gel volume of acetone and the gel was this time left for at least 30 minutes. This procedure was then repeated 2 to 3 times until the gel was shrunken into a white powder. The gel was finally washed on a glass filter with acetone and then dried in an oven (70° C.) over night. The ligand density was then measured using elemental analysis of the dried material. When calculating the amount of remaining uncoupled allyl groups (FIG. 5B) the ligand density of the coupled arginine gel (mmol/g coupled gel) was adjusted for the added weight from the coupled arginine. This was done to be able to compare it with the allyl amount on the starting allylated gel (mmol allyl/g allylated gel). The amount of uncoupled allyl groups will then be the difference between the starting amount of allyl groups and the adjusted ligand density after coupling.

It should be noted that allylation is normally measured in micromole per milliliter (μmol/ml) on wet swollen (in water) carrier bead gel while ligand density of the final microcarrier is measured on dried gel in mmole/g units (Table 3). The degree to which carrier beads swell appears to be related to many factors including solution, temperature, as well as ligands and ligand densities. However in general swelling factors for bead volumes on going from dried to hydrated swollen state, such as used to calculate the data in FIG. 5A-5C and which include errors related to packing void volumes, ranged between 16 and 22 ml/g for the microcarriers of the invention (swollen in 0.9% NaCl). For the allylated gels, if they were dried, the swelling factor in general ranged between 11 to 17 ml/g (swollen in water).

### Functional Testing for Cell Culture

### A. Vero and MDCK Cells

### Evaluation of Cell Growth Ability

The microcarrier prototypes were tested for growth of Vero (African green monkey kidney epithelial) and MDCK (Madin Darby canine kidney epithelial) cells (see below). As positive controls and to allow the comparison of different experiments CYTODEX™ 1 and CYTODEX™ 3 were used as reference carriers in each test.

### Cell Lines and Cultivation Medium

MDCK cells were derived from ATCC (American Type Culture Collection) (Nr. CCL 34) and adapted to serum free growth.

During routine culture the cells were grown in DMEM/Ham's F12 (1:1) (Biochrom, Berlin, Germany) supplemented with 4 mM L-glutamine (Sigma Aldrich, Austria), 0.1% soy peptone (HYPEP™ 1510, Quest, Naarden, the Netherlands) 0.01% β-Cyclodextrin (Roquette, Lestrem, France) and 0.01% protein free additive (Polymun Scientific, Vienna, Austria).

For the last passage before the inoculation of microcarriers the cultivation medium was changed to OPTIPRO® (Invitrogen, Carlsbad, USA). Cultivation on microcarriers was done in the same medium, for inoculation 20% of conditioned OPTIPRO® was added.

Vero cells were derived from ATCC (Nr. CCL 81) and adapted to serum free growth. The cells were cultivated in DMEM/Ham's F12 (1:1) (Biochrom, Berlin, Germany) supplemented with 4 mM L-glutamine (Sigma Aldrich, Austria), 0.1% soy peptone (HYPEP™ 1510, Quest, Naarden, the Netherlands) and 0.01% β-Cyclodextrin (Roquette, Lestrem, France).

The microcarriers were hydrated and washed in Ca²⁺ and Mg²⁺ free PBS (Sigma Aldrich, Austria) and then sterilised by autoclavation. One day before inoculation the microcarriers were washed once with cultivation medium and transferred to the cultivation vessel for temperature and pH equilibration (37° C., 7% CO₂ in the atmosphere). All experiments were done in 125 ml Techne spinner flasks at a working volume of 60 ml. To prevent sticking of the carriers to the glass the pyrogen free flasks were siliconised using SIGMACOTE® (Sigma Aldrich, Austria) and then sterilised by autoclavation.

Inoculum for MDCK cell tests was propagated in t-flasks (Nunclon, Nunc, Roskilde, Denmark). For cell harvest each t-flask was washed with PBS and the cells detached with 2 ml TRYPLE™ (Invitrogen, Carlsbad, USA). After incubation at 37° C. for 20 to 30 minutes, the detached cells were pooled and centrifuged (200 g for 10 min) to remove the proteolytic enzyme. The pellet was resuspended in OPTIPRO® (Invitrogen, Carlsbad, USA). The concentration of the detached cells was determined in a haemocytometer (Neubauer improved). Cell viability was analysed by the trypan blue exclusion method. The amount of cell suspension to reach a concentration of 2×10⁵ viable cells/ml in the final volume of 60 ml was calculated and the inoculum added to the equilibrated spinner flasks. Conditioned OPTIPRO® medium was added to a final concentration of 20% and the volume brought to 60 ml with OPTIPRO®. The flasks were then put to continuous stirring at 50 rpm in a 37° C. warm room.

Inoculum for Vero cell tests was prepared in T175 flasks (Nunclon, Nunc, Roskilde, Denmark) or R850 roller bottles (CELLBIND® 850 cm², Corning Life Sciences, Schipol Rjik, the Netherlands). For seeding of microcarrier cultures the cells were detached with EDTA (0.02% in PBS without Ca²⁺ and Mg²⁺). After washing of the cell layer with PBS the EDTA solution was added (2 ml for T175 flasks, 10 ml for R850 bottles) and the vessels were incubated at 37° C. for 20 to 30 minutes. The detached cells were then pooled and diluted in cultivation medium. Cell concentration and viability was determined as described for MDCK cells. The amount of cell suspension to reach a concentration of 2×10⁵ viable cells/ml in the final volume of 60 ml was calculated and the inoculum added to the equilibrated spinner flasks. The volume was brought to 60 ml with cultivation medium and the flasks were put to continuous stirring at 50 rpm in a 37° C. warm room.

During the cultivation daily samples were taken to determine metabolite concentrations (glucose, lactate, glutamine and glutamate). Media changes were done as required to keep the residual glucose concentration above 1 g/l and prevent nutrient limitation.

### Cell Counting and Microphotography

Daily samples were taken to determine cell number and morphology. For cell counting 1 ml carrier suspension was removed from the spinner flask and transferred to a test tube. When the carriers had settled the supernatant was removed and the carriers were resuspended in 1 ml lysis buffer (0.1% crystal violet in 0.1 M citric acid). After a minimum incubation period of 1.5 h the released nuclei were counted using a microscope and a haemocytometer. Data about the cell concentration were used to calculate the cell growth rate and cell attachment. The cell attachment was measured six hours after inoculation and was calculated as cell concentration on the microcarriers divided by the viable cell concentration used for inoculation.

For microphotography the cells on the CYTODEX™ carriers were fixed and stained with haematoxilin. The staining solution consists of 0.9 g haematoxilin, 0.18 g NaIO₃, 15.45 g AlK(SO₄)₂×12H₂0, 45 g Chloralhydrate and 1 g Citric acid mono hydrate in 1 liter RO water. Haematoxilin and Chloralhydrate were obtained from Carl Roth GmbH, Karlsruhe, Germany, all other chemicals from Sigma Aldrich. The carriers were viewed at 100 fold magnification.

### B. Human Mesenchymal Stem Cells

Human mesenchymal stem cells (hMSCs) were tested as these cells are of human origin, and quite different from MDCK or Vero cells. MSCs can show very different growth characteristics on variety of surfaces (see Biomaterials Vol. 29, pp. 302-313, 2008. Assessment of stem cell/biomaterial combinations for stem cell-based tissue engineering, Sabine Neuss et al.) and are of obvious biomedical significance. In regard to the latter hMSCs represent cells whose culture is often directed to using the cells as a product, e.g. for cell therapy or high throughput cell based screening. This is fundamentally different than in the case of Vero or MDCK cells for vaccine production or CHO cells for recombinant protein where the cells produce the target product.

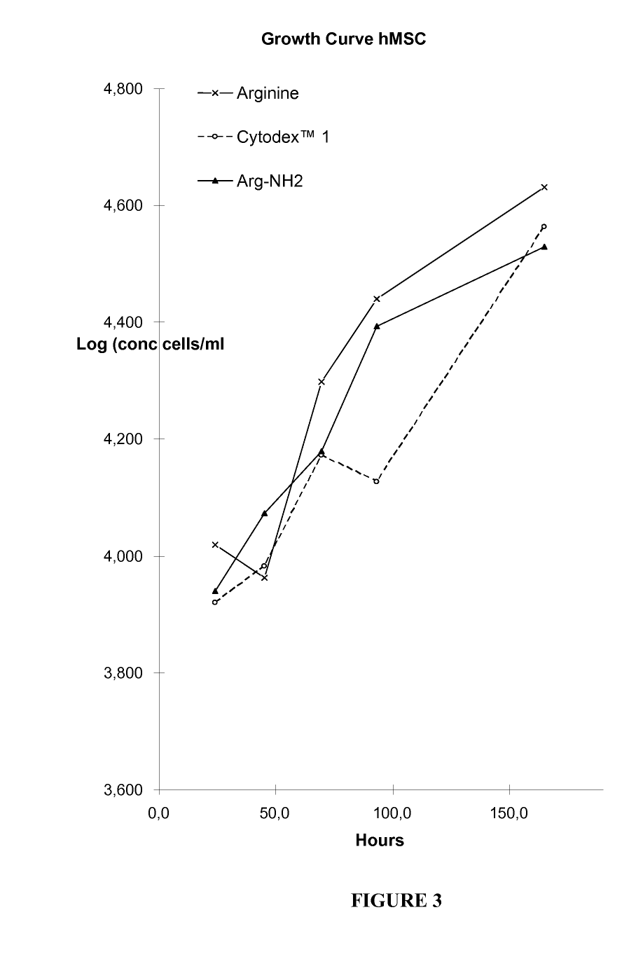

MSC culture was performed in microtitre plates under conditions more amenable to further use of the cells for high throughput screening. Prototype cell carriers were evaluate against commercial CYTODEX™ carriers in regard to three parameters a. cell growth, b. cell morphology and general healthy appearance, and c. ease of removal of the cells. Results are given in FIG. 3 and Table 3.

TABLE 2
Materials and Methods

| | Article | Lot | Vendor/ |
|---|---|---|---|
| Materials | number | number | distributor |
| DMEM with GlutaMAX | 31966 | 12553 | GIBCO |
| Human mesenchymal stem | PT-2501 | 6F4085 | Lonza |
| cells (hMSC) | | | |
| Hepes 1M | 15630-049 | 61734A | GIBCO |
| Phosphate buffered | BE17-512F | 6MB0103 | Lonza |
| saline (PBS) 0.0095M | | | |
| PO₄ (Ca²⁺, Mg²⁺-free) | | | |
| EDTA | E6758-100G | 085K00291 | Sigma |
| PBS/EDTA 0.02% | E8008 | 097K2408 | Sigma |
| Mesenchymal stem cell | PT-3238 | 01112285 | Lonza |
| basal media, MSCBM | | 08105549 | |
| Mesenchymal cell Growth | PT-4106E | 08104072 | Lonza |
| supplement, MCGS * | | 08105451 | |
| L-Glutamine * | PT-4107E | 08104173 | Lonza |
| | | 08105452 | |
| Penicillin/ | PT-4108E | 08104174 | Lonza |
| Streptomycin * | | 08105496 | |
| * Included in Single | PT-4105 | 08104175 | Lonza |
| quots | | 08105549 | |
| Trypsin/EDTA | CC-3232 | 01111734 | Cambrex/ |
| | | | In Vitro AB |
| Trypan blue | | U1743:027 | Christine |
| | | | Sund- |
| | | | Lundström |
| CYTODEX ™ 1 | 17-0448-01 | 310919 | GE Healthcare |
| CYTODEX ™ 2 | 17-0484-02 | 288234 | GE Healthcare |
| CYTODEX ™ 3 | 17-0485-01 | 303810 | GE Healthcare |
| SEPHADEX ™ G-50 | U1661008/2 | — | — |
| SEPHADEX ™ G-50 F för | 30-1525-00 | 10007610 | GE Healthcare |
| CYTODEX ™ | | | |
| Varioklav L7AK201 | — | IP 26473 | — |
| Heracell 150 incubator | — | IP 28214 | Bergman |
| | | | Labora |
| Centrifuge, Multifuge3 | — | IP 21567 | Heraeus |
| S-R | | | |
| Sarstedt 15 ml sterile | 62554502 | — | Sarstedt |
| test tube | | | |
| Falcon 50 ml sterile | 352070 | — | Falcon |
| test tube | | | |
| Falcon 15 ml sterile | 352090 | — | Falcon |
| test tube | | | |
| 24-well polystyrene | 144530 | 089864 | Nunc |
| microtitre plates | | | |
| Bürker hemocytometer | 013-2290 | — | Bergman |
| | | | Labora |
| Hematoxylin | MHS32-1L | 016K4359 | Sigma |
| Hematoxylin | GHS132-1L | 116K4350 | Sigma |

Preparation of Medium for Human Mesenchymal Stem Cells (hMSC)

Aseptically open bottle of mesenchymal cell growth supplement, MCGS, add contents to 440 ml bottle of mesenchymal stem cell basal medium, MSCBM. Add entire amount from each cryovial of L-Glutamine and Penicillin/Streptomycin to the MSCBM. The medium, with all additives included, is named mesenchymal cell growth medium, MSCGM.

### Thawing of Cells/Initiation of Culture

All cell culture work is performed in sterile field, such as a linear air flow (LAF) bench and with sterile technique. Add cell medium to a suitable T-flask and allow equilibrating at 37° C., 5% CO₂ for at least 30 minutes. Thaw cryovial with cells in a 37° C. water bath until all the ice melts (<3 minutes) and then remove the vial immediately. Add thawed cell suspension to a sterile 50 ml Falcon tube with 5 ml of room tempered medium. Centrifuge at 400 g for 5 minutes at room temperature. Resuspend cells in a suitable volume of the preheated medium. Add the cells to the T-flask; incubate at 37° C. and 5% CO₂. Media change after 3-4 days and subculture when 90% confluent.

### Sub Culturing:

Remove and discard medium from used T-flask. Wash attached cell layer with PBS containing 0.02% EDTA. Remove and discard the PBS/EDTA solution. Add Trypsin/EDTA solution to cover the cell layer. Incubate hMSC at room temperature for a few minutes. Then observe under a microscope. When >90% of the cells are rounded and detached, add equal volume of tempered medium to the flask. Do not incubate the cells with Trypsin/EDTA longer than 15 minutes. To remove the trypsin, centrifuge cells at 400 g for 5 minutes at room temperature. Resuspend the cell pellet in a suitable volume of preheated medium and count the cells. Count living cells using Trypan blue as follows. Add 20 μl of cell suspension +20 μl of Trypan blue and count all white cells (cells that have been coloured blue are dead cells). Recommended seeding density for hMSC is 5000-6000 cells cm². The hMSC cells had to be subcultivated once a week for three times before enough amount of cells were obtained.

Preparation of Micro Carriers for hMSC:

1 gram of dry CYTODEX™ commercial or prototype or control microcarriers were swollen in 50 ml PBS and 0.06-0.41 g of the prototypes (dry powder) were swollen in 5-10 ml of Ca²^{⁺}, Mg²^{⁺}-free PBS for 3 hours at room temperature with occasional gentle agitation. Approximately 1 ml settled gel from each sample was transferred to a 15 ml tube and 5 ml PBS was added and well mixed. This wash step was repeated four times. Between each wash the carriers were settled. Afterwards the microcarriers were autoclaved (20 minutes, 121° C.). Preparations of microcarriers were performed under sterile conditions after the sterilization. Before adding the cells, the microcarriers were equilibrated twice in basal medium with the same procedure as the washing step. After media removal from the last equilibrating step, 4 ml complete medium were added and the carriers were stored at +2-8° C.

Start of hMSC Culture.

The supernatant from the samples were removed and an equal volume of complete medium was added to get a 50% bead solution. Experiments typically included 25 samples, three positive controls and one negative control, one well for each sample, totally 29 wells (three plates). 800 μl medium and 40 μl of the bead solution was added/well in a 24 well plate. This corresponds to approximately 5000 beads/well. The plates were equilibrated at 37° C., 5% CO₂ for at least 1 hour. After that 125 μl cell suspension (40000 cells/well) were added. Cells were incubated with the beads for 3 hours at 37° C. and 5% CO₂ and then the beads were transferred to new wells. This was done because some cells attach to the bottom of the wells, which made it more difficult to evaluate if the cells attached to the beads or not. Cell attachment and spreading were studied in the microscope at 7, 23 and 48 hours. Notes and photos were taken. Results are shown in Table 3 below.

After 48 hours a detachment test was done on one control and test samples. The beads were transferred to a tube, washed twice with PBS. Centrifuged at 200 g for 5 minutes at room temperature and then 0.5 ml Trypsin/EDTA was added. The beads were transferred to a micro titer plate and inspected by microscope as regards cell detachment. Results are shown in Table 3.

After 120 hours a new detachment experiment was done at the other two controls and samples. The beads were transferred to a tube, washed once with PBS/EDTA 0.02% and 0.5 ml Trypsin/EDTA was added. The beads were transferred to a micro titer plate and inspected by microscope to evaluate cell detachment. The beads settled without centrifugation so that step was excluded. Results are shown in Table 3.

In some cases similar experiment was followed however cells were cultured up to 72 hours and evaluated at 4, 24, 48 and 71 or 72 hours (instead of 7, 23 and 48 hours). In addition cells were allowed to grow on the beads for 144 hours and then tested for ease of removal using 0.02% EDTA in PBS, instead of just PBS, prior to normal trypsinization. Results shown in Table 3.

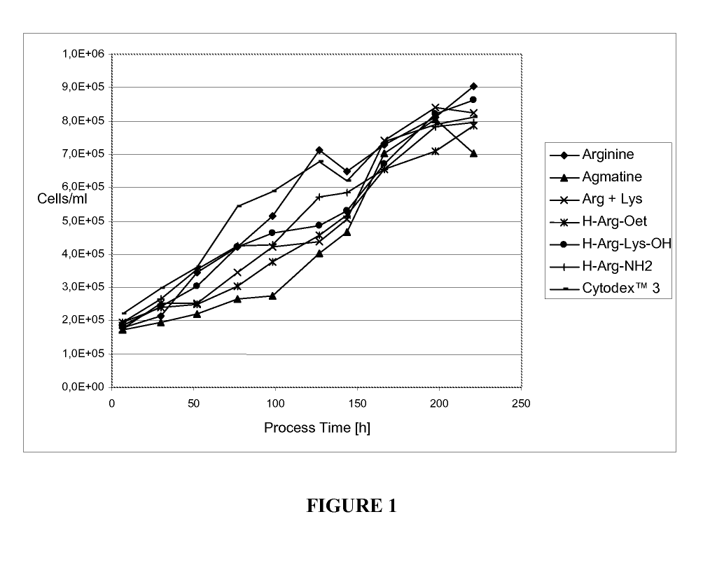

The cell growth abilities of the microcarriers with different ligands have been evaluated on Vero cells (using serum free conditions). FIG. 1 shows that the modified microcarriers produced according to the invention show comparable growth as conventional commercial cell growth media (CYTODEX™ 3).

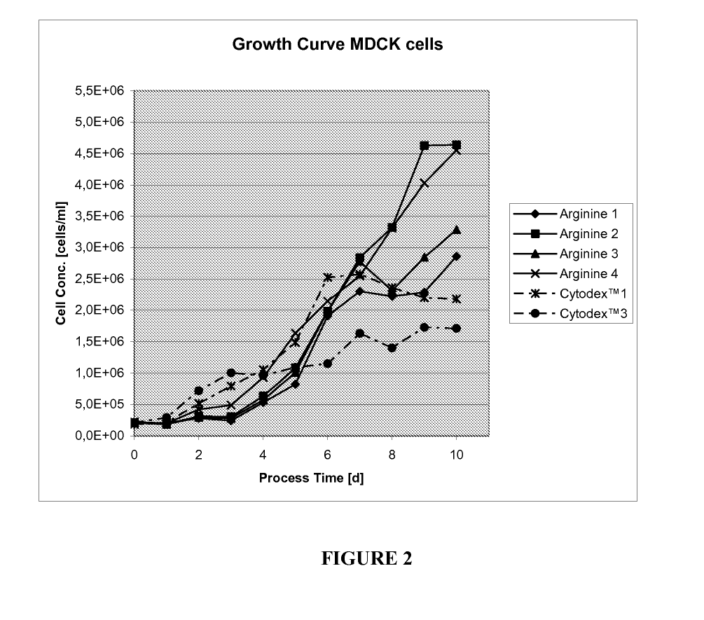

FIG. 2 shows the cell growth for MDCK cells on arginine-modified microcarriers produced according to the invention. Effectiveness of various ligands and relation of results to ligand type, density and activating allyl group density are generally in keeping with Vero cells.

FIG. 3 shows cell growth of human mesenchymal stem cells (hMSC's) on microcarriers produced according to the invention.

Effectiveness of various ligands and relation of results to ligand type, density and activating allyl group density (Table 3) are generally in keeping with the other cell types. Detachment experiments suggest the new carriers can offer ease of detachment equal to or better than CYTODEX™ commercial control carriers (Table 3).

TABLE 3
Culture and Removal of Human Mysenchymal Stem Cells from Cell Carriers in Microtitre Well Plates

| | Allyl | | | Cells | Cells | Cells | Cells | Cells | 48 h | 120 h | 144 h |
|---|---|---|---|---|---|---|---|---|---|---|---|
| | μmol/ | | Ligand | Adhere | Spread | Spread | Spread | Spread | Detach. | Detach. | Detach |
| Carrier | ml | Ligand | mmol/g | 7 h | 7 h | 23 h | 48 h | 72 h | (min) | (min) | (min) |
| U1661008/2 | 0 | none | 0 | 0 | 0 | 0 | 0 | ND | ND | ND | ND |
| CYTODEX ™ 1 | — | DEAE | — | +1 | 0 | +1 | +2 | ND | 19 | ND | >15 |
| CYTODEX ™ 2 | — | — | — | +1 | 0 | +1 | +1 | ND | ND | 9 | ND |
| CYTODEX ™ 3 | — | — | — | +2 | +1 | +2 | +3 | +4 | ND | 12 | >15 |
| U1972011 | — | DEAE | 2.93 | +1 | 0 | +1 | +1, U | ND | ND | ND | ND |
| U1972014 | ND | Q | ND | +1 | 0 | +1 | +1, U | ND | ND | ND | ND |
| U1662096 | 103 | Arg + Lys | ND | +1 | 0 | 0 | 0 | ND | ND | ND | ND |
| U1662096 | 153 | Arg + Lys | ND | +1 | +1 | +1 | +2 | ND | 19 | ND | ND |
| U1972022 | 125 | Arg | 0.88 | +1 | +1 | +2 | +2, U | ND | 19 | ND | ND |
| U1662079 | 153 | Arg | 1.13 | +1 | +1 | +1 | +2 | ND | ND | ND | ND |
| U1692051 | 170 | Arg | 0.89 | +1* | +1 | +2 | +3 | +4 | ND | ND | 15 |
| U1972013 | 257 | Arg | 0.91 | +1 | 0 | 0 | 0 | ND | ND | ND | ND |
| U1662086 | 103 | H-Arg-Arg- | 0.47 | +1 | 0 | +2 | +2, U | ND | ND | ND | ND |
| | | OH | | | | | | | | | |
| U1662080 | 103 | Lys | 0.69 | +1 | 0 | 0 | 0 | ND | ND | ND | ND |
| U1662081 | 153 | Lys | 1.02 | +1 | 0 | +1 | +1 | ND | ND | ND | ND |
| U1972021 | 125 | H-Arg-Lys- | 0.56 | +1 | +1 | +1 | +1 | ND | ND | 12 | ND |
| | | OH | | | | | | | | | |
| U1662093 | 153 | H-Arg-Lys- | 0.59 | +1 | +2 | +2 | +2, U | ND | ND | ND | ND |
| | | OH | | | | | | | | | |
| U1662088 | 103 | H-Arg-NH2 | 1.06 | +1 | +2 | +2 | +3 | ND | 19 | ND | ND |
| | | 2HCl | | | | | | | | | |
| U1972023 | 125 | H-Arg-NH2 | 0.70 | +1 | 0 | +1 | +1, U | ND | ND | ND | ND |
| | | 2HCl | | | | | | | | | |
| U1662096 | 256 | H-Arg-NH2 | 0.68 | +1 | 0 | +1 | 0 | ND | ND | ND | ND |
| | | 2HCl | | | | | | | | | |
| U1789061 | 103 | H-Arg-Oet | 0.65 | +1 | 0 | +1 | +1 | ND | ND | ND | ND |
| U1789062 | 153 | H-Arg-Oet | 0.92 | +1 | +1 | +2 | +3 | ND | ND | 12 | ND |
| U2010013 | 170 | H-Arg-Oet | 0.80 | +1* | +1 | +1, U | +3, U | +3 | ND | ND | 10 |

CYTODEX ™ 1, 2, 3 are commercial media available from GE Healthcare, which use similar base matrix.
ND = not determined, Arg = arginine, Lys = lysine, Arg + Lys is equimolar mixture. DEAE = diethylaminoethyl, Q = quarternary amine. Result Scoring defined in text, 0 = none, + = detectable, +2 = significant, +3 = very good, +4 = excellent, U = uneven, with some bead to bead cell growth differences,
*refers to cell adherence observed at 4 instead of 7 hours.
Detachment minutes to significant visual detachment, 48 and 120 h culture times subjected to trypsin, 144 to EDTA and trypsin.

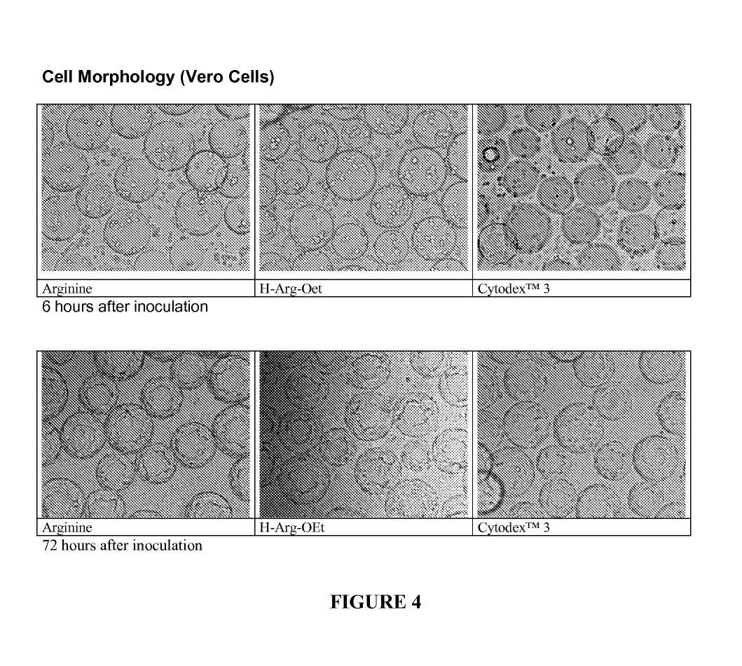

FIG. 4 shows cell morphology of Vero cells after attachment (6 h) and after 72 hours growth on arginine (Arg) and H-Arg-O-Et (Table 1) ligand modified, and commercial CYTODEX™ 3 microcarriers produced according to the invention.

FIG. 5A-5C compares some Vero cell growth data in terms of arginine ligand density, unreacted residual allyl groups (which are expected to further hydrolyse under reaction conditions) and the ratio of arginine ligand density to unreacted allyl groups. For ease of comparison allyl and arginine ligand density have been expressed in mmol/g (see above comments regarding assays and swelling factors). It can be seen that for arginine ligand the ligand density should be above 0.5 mmol/g dry gel to make the microcarriers of the invention able to best support cell growth. Too low a ligand density, coming from using a gel with too low starting allyl content or a low yield in the coupling reaction, will make the microcarriers of the invention less able to support cell growth.

According to the invention it is also very important that the ratio of coupled ligand (here exemplified by arginine) to the starting allyl content is correct since this can have a crucial impact on the cell growth (see FIGS. 5B and 5C). If this ratio (FIG. 5C) is kept high cell growth is optimal. One possible explanation for this is that remaining uncoupled allyl groups are converted to glycerols during the reaction, with the presence of such surface hydroxyl groups having a negative effect on cell growth. This means that keeping a high yield of the coupling reaction is necessary to obtain the microcarriers of the invention and thus a ratio of ligand coupled allyl to uncoupled allyl above 1.5 is preferred for the microcarriers of the invention. Naturally one might expect the actual ‘threshold’ ligand concentration to vary somewhat with base matrix carrier, ligand type, cell type, culture media, culture conditions, etc. Nevertheless similar results may be seen with other ligands and cell types (e.g. hMSC data in Table 3 Arg-Arg ligand). This suggests that microcarriers made with a too high starting allyl level may not be able to support optimal cell growth, even if the yield in the coupling reaction is good and a high ligand density is obtained, since the amount of remaining allyls (converted into glycerols) can still be too high. The remaining level should thus be kept below 0.6 mmol/g to afford optimal cell growth.

In summary the conditions to achieve optimal cell growth for Vero or MDCK cells is a) a ligand density of above 0.5 mmol/g of dry gel, b) a remaining uncoupled allyl level of below 0.6 mmol/g and c) a ratio of coupled ligand to uncoupled allyl of above 1.5. To afford growth of Vero cells at cell densities useful for various applications all of the above stated conditions should be fulfilled see FIG. 5. The optimal conditions may vary depending on surface matrix, ligand, cell type and culturing conditions. However carriers meeting these conditions were also suitable for culture of other varied cell types such as MDCK and hMSC. It should be obvious that if a carrier or similar surface was activated with allyl reagent but further modified with cell binding arginine or other ligand in a pattern it should be possible to achieve patterned cell culture.

### Virus Productivity

Influenza Infection and Determination of Virus Yield

Virus infection of the microcarrier cultures was done after cells on the reference carriers reached confluence. Influenza virus A Singapore/57 (H2N2), lot S0007-230306 was added at a MOI of 0.01. The culture was supplemented with trypsin at a concentration of 1 μg/ml. The virus containing supernatant was harvested after four days of cultivation at 33° C. when full cytopathic effect was visible.

Virus concentration was determined by a haemagglutination (HA) test. The sample was centrifuged for 10 min at 3000 g to remove cell debris. In a microtiter plate a 1:2 dilution series of each sample was prepared in PBS. 50 μl of the dilutions were used for the HA test. PBS was used as negative control, freshly thawed influenza standard (NIBSC, Hertfordshire, UK) was used as a reference. 50 μl of human erythrocytes in PBS (0.5%) were added to each well and the plate incubated at room temperature. After the erythrocytes in the control wells had settled (90 to 120 min) the test was evaluated. The highest dilution with complete haemagglutination was determined for each sample and defined as containing one HA unit per 50 μl of diluted sample.

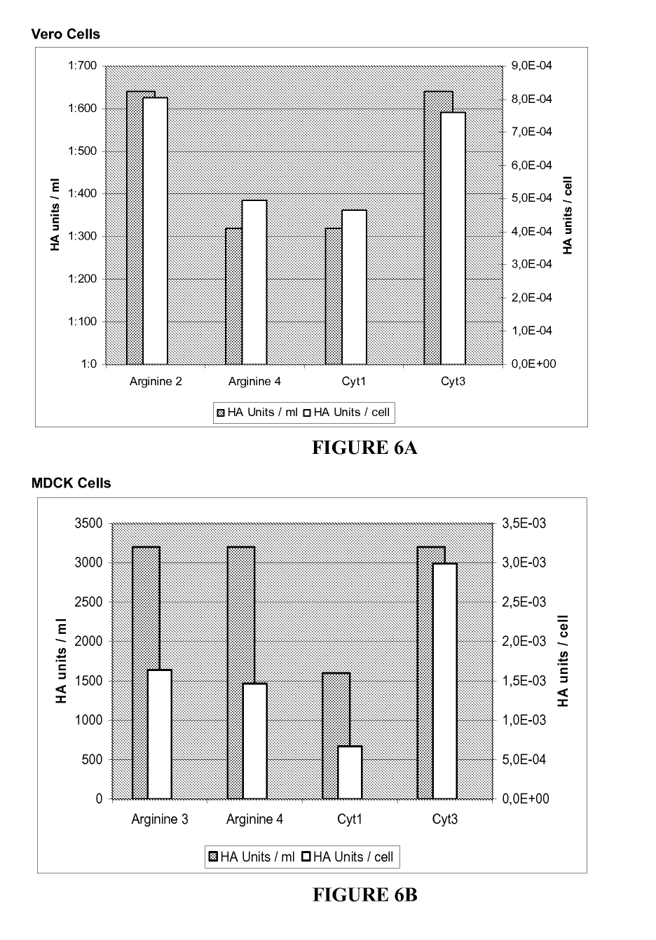

The arginine-modified microcarriers' virus productivity for both Vero and MDCK cells was compared to commercial CYTODEX™ microcarriers using a standard haemagglutination (HA) test. As can be seen in FIG. 6 the novel microcarriers give a higher virus productivity for both MDCK and Vero cells compared to CYTODEX™ 1 and comparable productivity to CYTODEX™ 3 for Vero cells. Again it should be noted that CYTODEX™ 3 contains a gelatin surface coating whereas, as with CYTODEX™ 1, the equally performing novel arginine based carriers only had simple ligand modified surfaces.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

What is claimed is:

1. A method for production of a cell attachment and culture surface comprising a biocompatible guanidine group-containing ligand, wherein the ligand is coupled via reaction involving a primary amine to the surface which is activated by activation groups such that the final molar ratio of grafted ligand and ungrafted activation groups is above 1.5.

2. The method of claim 1, wherein the cell culture surface is a microcarrier based on a natural polymer, such as dextran, starch and cellulose.

3. The method of claim 1, wherein the ligand density is above 0.5 mmol/g cell culture surface and remaining activation groups after coupling is less than 0.6 mmol/g cell culture surface.

4. The method of claim 1, wherein the ligand is arginine, agmatine, guanosine, guanidine or adenosine, or derivatives thereof and combinations thereof.

5. The method of claim 1, wherein the ligand comprises a dipeptide including at least one arginine.

6. The method of claim 1, wherein the cell culture surface is activated by activation groups selected from allyl, epoxide or glycidoxyl groups.

7. The method of claim 1, wherein the surface or microcarrier is coated with an animal protein-free coating.

8. The method of claim 1, wherein the microcarrier provided with magnetic particles.

9. The method of claim 1, wherein the microcarrier is provided with an imaging (e.g. fluorescent or radioactive) agent.

10. The method of claim 1, wherein the microcarrier is made of biodegradable material.

11. The microcarriers produced according to the method of claim 1.
